(19) United States
(12) Patent Application Publication
(10) Pub. No.: US 2024/0208830 A1
(43) Pub. Date: Jun. 27, 2024

(54) METHOD FOR PRODUCING LARGE-DIAMETER SIO2 POWDER, AND COSMETIC COMPOSITION CONTAINING SAME

(71) Applicant: SUKGYUNG AT CO., LTD., Ansan-si, Gyeonggi-do (KR)

(72) Inventors: Hyung Sup LIM, Ansan-si, Gyeonggi-do (KR); Hyung Jun LIM, Anyang-si, Gyeonggi-do (KR); O Sung KWON, Gunpo-si, Gyeonggi-do (KR); Sang Gon KIM, Seoul (KR); Hyeon Seok CHOI, Ansan-si, Gyeonggi-do (KR)

(73) Assignee: SUKGYUNG AT CO., LTD., Ansan-si, Gyeonggi-do (KR)

(21) Appl. No.: 17/909,788

(22) PCT Filed: Mar. 3, 2021
(86) PCT No.: PCT/KR2021/002592

§ 371 Date: Sep. 7, 2022

(30) Foreign Application Priority Data
Mar. 27, 2020 (KR) 10-2020-0037569

(51) Int. Cl. C01B 33/193; A61K 8/02; A61K 8/25; A61Q 19/10

(52) U.S. Cl. CPC C01B 33/193; A61K 8/025; A61K 8/25; A61Q 19/10; A61K 2800/28; C01P 2004/32

(57) ABSTRACT

The present invention pertains to a method for producing SiO₂ powder having a particle size of 1.0-20.0 μm, the method comprising the steps of: adding CH₃COOH to H₂O and stirring; after the stirring, adding TEOS and stirring; removing the synthetic stock solution in a filter press, and washing the solution with H₂O; and after the washing, drying the solution to obtain dried silica particle powder having a moisture content of 0.5% or less. Also provided is monodispersed amorphous spherical silica particle powder having a particle size of 1.0-20.0 μm, a whiteness of at least 90, and a purity of at least 99.9%. Large-diameter silica particles of the present invention are monodispersed particles having similar particle sizes, are spherical and have a smooth surface, can be used as cosmetic scrubs due to having a soft feeling of use without causing skin irritation, and have the advantage of being environmentally friendly.

### TECHNICAL FIELD

The present invention disclosed herein relates to large-diameter silica particles, and a cleansing cosmetic composition containing the same.

### BACKGROUND ART

The cleansing cosmetic contains a scrubbing agent that exfoliates the stratum corneum by physical action. As the scrubbing agent, microscopic plastic such as a resin has been used, and the plastic particles easily absorb chemical substances such as pesticides and are lightweight, and thus it is difficult to remove the plastic particles from the sewage treatment plant. Accordingly, the plastic particles flow into rivers, oceans, ponds, swamps, and the like, and are accumulated in fish and shellfish. Through such fish and shellfish, the plastic particles may have an influence on human bodies.

Therefore, there is a need to develop a scrub material that reduces environmental pollution and has less irritations to the skin.

### DISCLOSURE OF THE INVENTION

### Technical Problem

The present invention provides a method for producing large-diameter SiO₂ powder which can be used as scrubs contained in a cosmetic composition, and a cosmetic composition containing the same as scrubs.

### Technical Solution

The present invention is characterized by providing a method for producing large-diameter SiO₂ powder having a particle size of 1.0-20.0 μm.

The production method is characterized by including the steps of:
adding CH₃COOH to H₂O and stirring;
after the stirring, adding TEOS and stirring;
removing the synthetic stock solution in a filter press, and washing the solution with H₂O; and
after the washing, drying the solution to obtain dried silica particle powder having a moisture content of 0.5% or less.

H₂O was used as a solvent, and CH₃COOH was added as a catalyst and stirred. Thereafter, TEOS was added and stirred. The longer the stirring time is, the larger the particles are generated. The synthetic stock solution was removed in the filter press, and washed with H₂O. Thereafter, drying was performed in a box dryer at 90° C. for 24 hours. Silica particles in a dried powder state was identified, and the moisture content was identified to be 0.5% or less by sieving in a 100-mesh sieve.

The production method is characterized in that TEOS:CH₃COOH:H₂O are each reacted at a 1:4:4 molar ratio.

The particles produced by the production method have monodispersed, amorphous, and spherical characteristics having a particle size of 1.0-20.0 μm, a whiteness of at least 90 and a purity of at least 99.9%.

More preferably, the particles are characterized in that the particle size is 2.0-10.0 μm.

Meanwhile, the present invention is characterized by providing a cosmetic composition containing, as scrubs, the silica particle powder of the present invention. The silica particle powder of the present invention has monodispersed, amorphous, and spherical characteristics having a particle size of 1.0-20.0 μm, a whiteness of at least 90, and a purity of at least 99.9%, and has smooth surfaces, and thus has good flowability and oil absorption rate, and therefore can be used as scrubs which are free from skin irritation and environmentally friendly in a cosmetic composition.

### Advantageous Effects

Particles of the present invention have characteristics in that silica particles according to the present invention are white powder, a particle size is 1.0-20.0 μm, a whiteness is >90, and a purity is >99.9%.

In addition, the large-diameter silica particles of the present invention are monodispersed particles having similar particle sizes, are spherical and have smooth surfaces, can be used as cosmetic scrubs due to having a soft feeling of use without causing skin irritation, and have the advantage of being environmentally friendly.

### BRIEF DESCRIPTION OF THE DRAWINGS

FIG. 1 is an SEM photograph of silica particles having a size of 3 μm obtained according to Example 1.

FIG. 2 is an SEM photograph of silica particles having a size of 5 μm obtained according to Example 2.

FIG. 3 is a particle size distribution graph of silica particles having a size of 3 μm obtained according to Example 1.

FIG. 4 is a particle size distribution graph of silica particles having a size of 5 μm obtained according to Example 2.

FIG. 5 is an X-ray diffraction diagram of silica particles having a size of 3 μm obtained according to Example 1.

FIG. 6 is an X-ray diffraction diagram of silica particles having a size of 5 μm obtained according to Example 2.

### MODE FOR CARRYING OUT THE INVENTION

Conventionally, silica particles have been produced using TEOS, water, and ethanol, but the present invention is characterized in that large-diameter silica particles are produced using TEOS, water, and acetic acid as raw materials.

Hereinafter, the present invention will be described in more detail with reference to examples.

### Example 1: Production Process of 3-μm Large-Diameter SiO2

In a reaction tube, the reaction is carried out at a ratio of TEOS:CH₃COOH:H₂O=1 mole:4 mole:4 mole. H₂O was used as a solvent, and CH₃COOH was added as a catalyst and stirred for 2 minutes. Thereafter, TEOS was added and stirred for 2 minutes. The synthesized stock solution was removed in the filter press, and washed with H₂O. Thereafter, drying was performed in a box dryer at 90° C. for 24 hours. Silica particles in a dried powder state was identified, and the moisture rate was identified to be 0.5% or less by sieving in a 100-mesh sieve.

### Example 2: Production Process of 5-μm Large-Diameter SiO2

In a reaction tube, the reaction is carried out at a ratio of TEOS:CH₃COOH:H₂O=1 mole:4 mole:4 mole.

CH₃COOH was added to H₂O and stirred for 4 minutes. Thereafter, TEOS was added and stirred for 2 minutes. The synthesized stock solution was removed in the filter press, and washed with H₂O. Thereafter, drying was performed in a box dryer at 90° C. for 24 hours. Silica particles in a dried powder state was identified, and the moisture rate was identified to be 0.5% or less by sieving in a 100-mesh sieve.

### Identification of Physical Properties of Large-Diameter Silica Particles

The silica particles produced according to Examples 1 and 2 above were identified to have the following physical properties.

### 1. Spherical Morphology

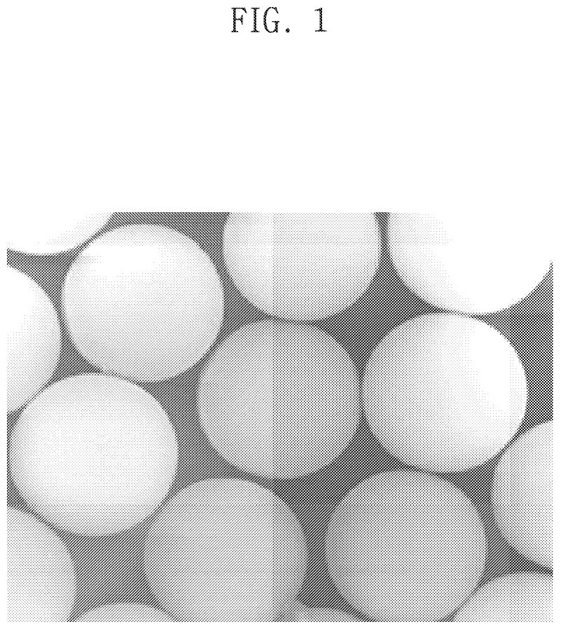

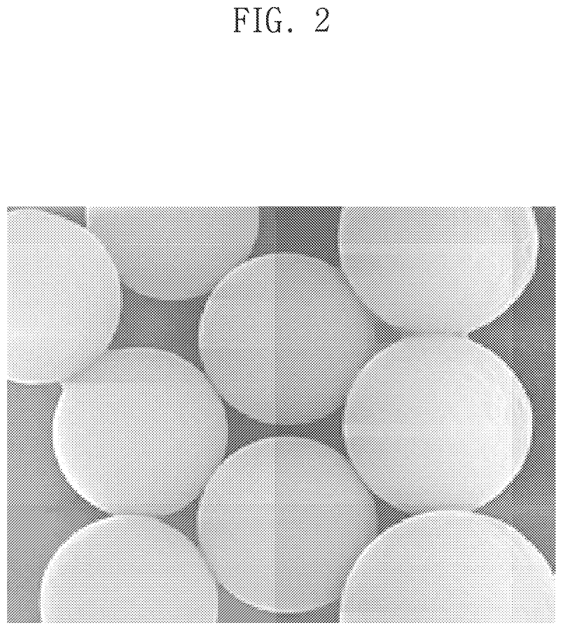

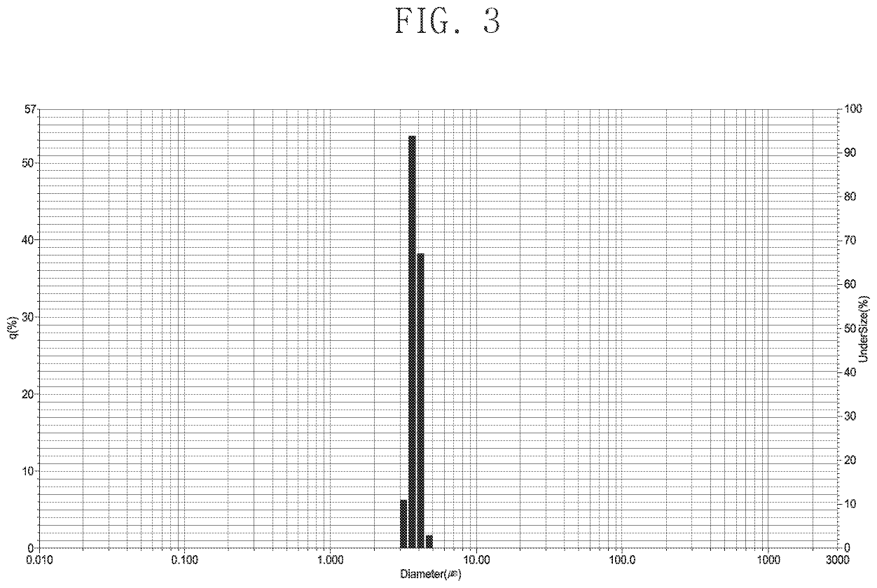

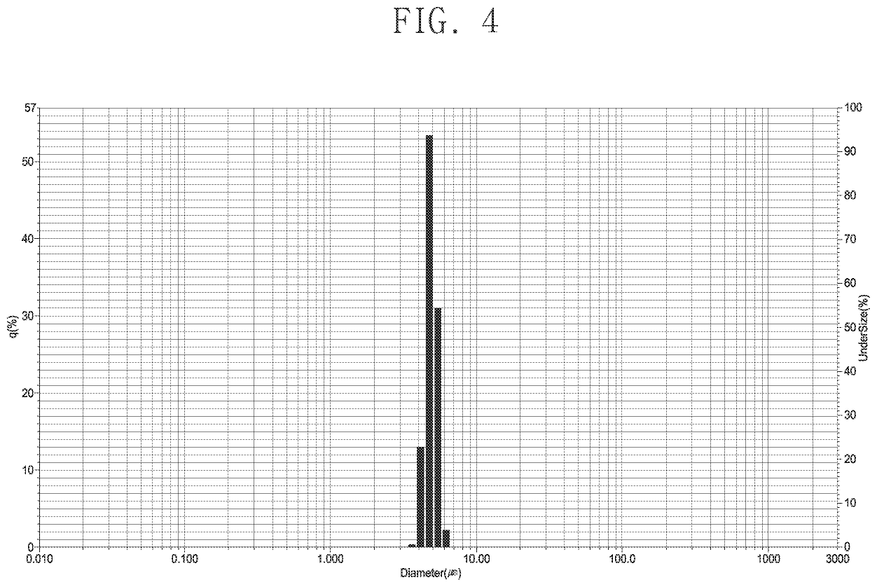

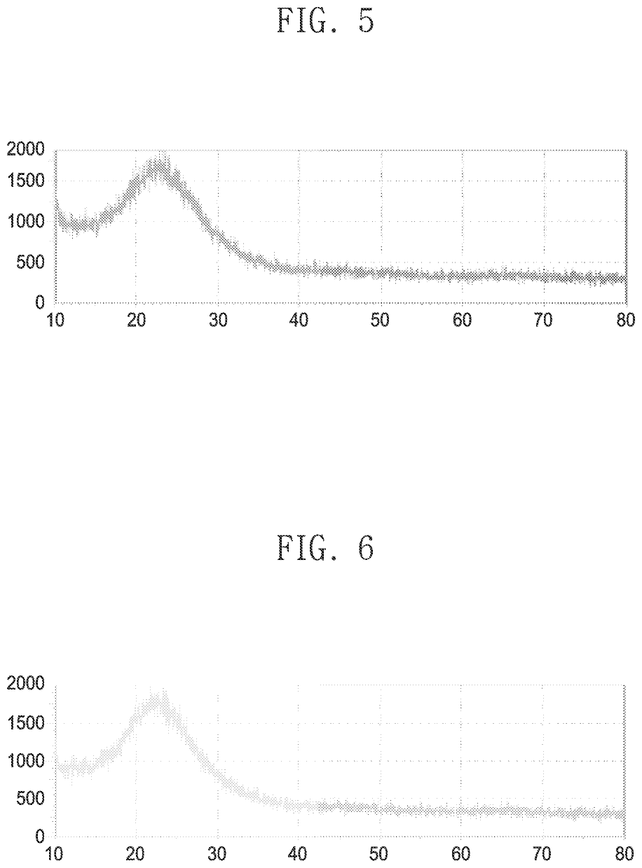

As shown in FIGS. 1 to 6, the particle size is 1.0-10 μm, and the produced silica particles are monodispersed spherical particles with a smooth surface having almost the same particle size.

### 2. Safety Amorphous Silica

As shown in FIGS. 1 to 6, the produced particles are amorphous silica.

### 3. Extreme White Powder

As shown in FIGS. 1 to 6, the produced silica particles have the characteristics of white powder having a whiteness of >90 or more, and a purity of >99.9%.

### Example 3: Cosmetic Composition Containing Large-Diameter SiO2

A cosmetic composition was produced as a cleanser containing, as a scrub, 1% to 15% of silica particles having the size of 3 μm and 5 μm and the above physical properties produced according to Examples 1 and 2.

When users washed their face with the cosmetic composition, users had less skin irritation. The silica particles of the present invention were used as scrub particles, and cosmetic active ingredients were mixed as a commonly used cleanser.

Comparative Example 1: a cleanser was prepared which contains a scrub of plastic particle ingredients such as a resin having the same content.

### Evaluation of Skin Spreadability/Skin Irritation/Skin Suppleness

The cosmetic composition of Example 3 was subjected to use tests, and the results are shown in the table. After 10 women washed their face, the sample of Example above was evenly massaged on the face, an aqueous emulsion was applied thereon, and skin spreadability, skin irritation, and skin suppleness were evaluated on a 5-point scale, and the average value was calculated.

The 5-point scale is as follows:
1: Very dissatisfied
2: Dissatisfied
3: Neutral
4: Satisfied
5: Very satisfied

TABLE 1

| | | Comparative |
|---|---|---|
| Evaluation item | Example 3 | Example |
| Skin spreadability | 4.2 | 3.7 |
| Skin irritation | 4.5 | 3.5 |
| Skin suppleness | 4.3 | 3.8 |

As above, the cosmetic composition containing particles of the present invention shows satisfaction higher than neutral compared to Comparative Example 1. Although described above with reference to the preferred embodiment of the present invention, those skilled in the art may variously modify the present invention within the scope without departing from the spirit of the present invention described in the following claims.

1. A method for producing SiO₂ powder having a particle size of 1.0-20.0 μm, the method comprising the steps of:
adding CH₃COOH to H₂O and stirring;
after the stirring, adding TEOS and stirring;
removing the synthetic stock solution in a filter press and washing the solution with H₂O; and
after the washing, drying the solution to obtain dried silica particle powder having a moisture content of 0.5% or less.

2. The method of claim 1, wherein TEOS:CH₃COOH:H₂O is reacted at a molar ratio of 1:4:4.

3. Monodispersed amorphous spherical silica particle powder having a particles size of 1.0-20.0 μm, and a whiteness of at least 90 and a purity of at least 99.9%.

4. The silica particles powder of claim 3, wherein the particle size is 3.0-5.0 μm.

5. A cosmetic composition containing the silica particle powder of claim 3.
